(19) United States
(12) Patent Application Publication
(10) Pub. No.: US 2019/0120822 A1
(43) Pub. Date: Apr. 25, 2019

(54) METHODS AND SYSTEMS FOR DRUG DISCOVERY AND SUSCEPTIBILITY ASSAY IN USING A FERROFLUID

(71) Applicant: ANCERA, INC., Branford, CT (US)

(72) Inventor: Hur KOSER, Wellingford, CT (US)

(21) Appl. No.: 15/982,926

(22) Filed: May 17, 2018

Related U.S. Application Data
(63) Continuation of application No. 14/777,507, filed on Sep. 15, 2015, now abandoned.
(60) Provisional application No. 61/798,458, filed on Mar. 15, 2013.

(51) Int. Cl. G01N 33/50; B01L 3/00; C12M 1/02; C12M 3/00; C12M 3/06; C12Q 1/02; C12N 13/00; B01L 7/04; G01N 1/40; B03C 1/32; B03C 1/28

(52) U.S. Cl. CPC G01N 33/5008; B01L 2200/0668; C12M 41/18; C12M 23/42; C12M 23/16; C12Q 1/02; C12N 13/00; B01L 7/04; B01L 3/502753; G01N 1/40; B01L 3/502715; B01L 3/502761; B03C 1/32; B03C 1/288; B01L 2200/0647; B01L 2300/0627; B01L 2300/1883; B01L 2300/0864; G01N 2001/4038; B03C 2201/26; B01L 2300/0636; B01L 2300/0877; B01L 2400/043; B01L 2400/0487; B01L 3/50273

(57) ABSTRACT

A system for determining drug effectiveness on a plurality of cells is described. The system includes flowing a ferrofluid mixed with a plurality of biological cells through an inlet portion of a cartridge, the cartridge comprising a plurality of microfluidic channels, the inlet is in communication with a portion of each of the plurality of channels, applying a magnetic field proximate at least one of the inlet portion and the plurality of micro-channels, wherein the magnetic field is configured to apply an indirect force on the mix, separating biologic cells according to at least a first type as the mix flows in a first direction; and directing at least the first type of cells toward a first sensor functionalized with receptors via at least one of the micro-channels, the sensor arranged proximate to a second portion of at least one of the micro-channels downstream from the first inlet portion.

### RELATED APPLICATIONS

This application claims benefit under 35 USC 119(e) of US provisional patent application no. 61/798,458, filed Mar. 15, 2013, and entitled, “Drug Susceptibility Assay in Biocompatible Ferrofluid” the entire disclosure of which is herein incorporated by reference in its entirety.

### FIELD OF THE DISCLOSURE

The present disclosure relates to bio-assays using ferrofluids, and in particular, to systems and methods for determining drug susceptibility of target cells in a biocompatible ferrofluid.

### BACKGROUND OF THE DISCLOSURE

During the course of drug discovery/development, it becomes necessary to determine the response of a population of cells to the candidate drug in order to determine the drug candidate's effectiveness as well as its side effects. This in vitro testing requires culturing the cells of interest in special media that contains the drug candidate. Cellular response could be slow (hours to days), and its characterization through rapid, easy-to-use systems is essential for efficient drug development.

Similarly, in diagnostics, it is critical to determine whether pathogens present in a sample from a patient are drug resistant or not before a targeted antibiotic regimen can be prescribed.

The crudest and most brute force way to determine drug susceptibility in a population of cells is to directly run a culture in the presence of the right dosage of that drug. If the cells keep growing in the culture as they should, they are deemed unresponsive or resistant to that drug. Especially in diagnostics, this approach leads to cultures that take days and present a significant obstacle to accurate and targeted treatment. With sepsis (infection in the blood), for instance, every hour that the patient is not treated results in a 7% increase in probability of mortality. Since blood cultures typically take 48 hours at least, the doctors have to prescribe broad spectrum antibiotics in the absence of an accurate determination of the pathogen's identity and its susceptibility to regular antibiotics. While ethically correct, this behavior invariably contributes to the proliferation of drug resistant pathogens, especially at the hospitals.

The direct culture approach is useful only for those pathogens that can be cultured rapidly and with relative ease using standard culture media. For other pathogens (such as Borrelia that causes Lyme disease), culturing is painfully slow (weeks) and difficult (very specialized, proprietary media formulations are required), and does not contribute to the initial treatment decision.

Newer susceptibility assays rely on measuring the growth of a smaller population of cells or pathogens by using sensitive transducers, such as electrode-based impedance sensors, quartz crystal microbalance (QCM) or surface plasmon resonance (SPR) sensors.

The signal-to-noise ratio in such transducers enable a quicker determination as to whether the cells in culture are growing and dividing. However, the cells or pathogens of interest must first be isolated, purified and immobilized on the active surface of the transducer. Therein lies the main shortcoming of this approach. In diagnosing sepsis, for instance, rare pathogens from whole blood need to be isolated and captured on the sensor surface before the drug susceptibility assay may be run. It is this isolation and capture step that has complicated these sensor-based assays and limited their practicality.

### SUMMARY OF THE DISCLOSURE

This teachings of this disclosure are a further application and development of previous series of disclosures, including, for example PCT publication no. WO2011/071912 and WO2012/057878, the noted disclosures of which are all herein incorporated by reference in their entireties.

In some embodiments, a system for determining drug effectiveness on a plurality of cells is described. The system includes a cartridge comprising a plurality of microfluidic channels, an inlet portion for receiving a ferrofluid mixed with a plurality of biological cells to form a mix, the inlet in communication with a portion of each of the plurality of channels, magnetic field means provided proximate at least one of the inlet portion and the plurality of micro-channels, a sensor arranged proximate to a second portion of at least one of the micro-channels downstream from the first inlet portion, the sensor functionalized with receptors for binding with at least a first type of biological cell, wherein the magnetic field is configured to apply an indirect force on the biological cells in the mix to separate at least a first type of biological cell from the mix, and at least a first channel of the plurality of the micro-channels is configured to receive biological cells of the first type and direct the first type of cells to the sensor.

In some embodiments, a method for determining drug effectiveness on a plurality of cells is described. The method includes flowing a ferrofluid mixed with a plurality of biological cells through an inlet portion of a cartridge, the cartridge comprising a plurality of microfluidic channels, the inlet is in communication with a portion of each of the plurality of channels, applying a magnetic field proximate at least one of the inlet portion and the plurality of micro-channels, wherein the magnetic field is configured to apply an indirect force on the mix, separating biologic cells according to at least a first type as the mix flows in a first direction; and directing at least the first type of cells toward a first sensor functionalized with receptors via at least one of the micro-channels, the sensor arranged proximate to a second portion of at least one of the micro-channels downstream from the first inlet portion, wherein the first type of cells bind with the receptors on the sensor.

In some embodiments, one or more of the following may also be included:
separating comprises at least one of separating, focusing and concentrating.
the type of biological cell comprises a biological cell of a predetermined size, shape, weight and/or configuration.
thermal managing means surrounding at least one of the cartridge, the first micro-channel, and the remainder of micro-channels to substantially maintain the micro-channels at a first temperature.
at least one of the cartridge and the first micro-channel are configured to receive a first drug at a predetermined first dosage.
the sensor is configured to produce a signal determinative of the susceptibility of the first type of cells to the drug.
the signal corresponds to the cell growth rate of the first type of cells.
the sensor comprises an impedance sensor, and the system further comprises a controller having operating thereon computer instructions configured to track a signal of the sensor, such that an increase in impedance corresponds to an increase in the total cell volume of the first type of cells.
the sensor comprises a quart-crystal-microbalance (QCM), and the system further comprises a controller having operating thereon computer instructions configured to track a signal of the sensor, such that an increase in mass corresponds to an increase in the total cell volume of the first type of cells tracks changes in the total mass of the cells bound to the surface.

The above-noted embodiments, as well as other embodiments, will become even more evident with reference to the following detailed description and associated drawing, a brief description of which is provided below.

### BRIEF DESCRIPTION OF THE DRAWING

FIG. 1 is a schematic of a drug susceptibility assay system, according to some embodiments of the present disclosure.

FIG. 2 is an enlarged schematic of a drug susceptibility assay system according to some embodiments of the present disclosure.

### DETAILED DESCRIPTION OF SOME OF THE EMBODIMENTS

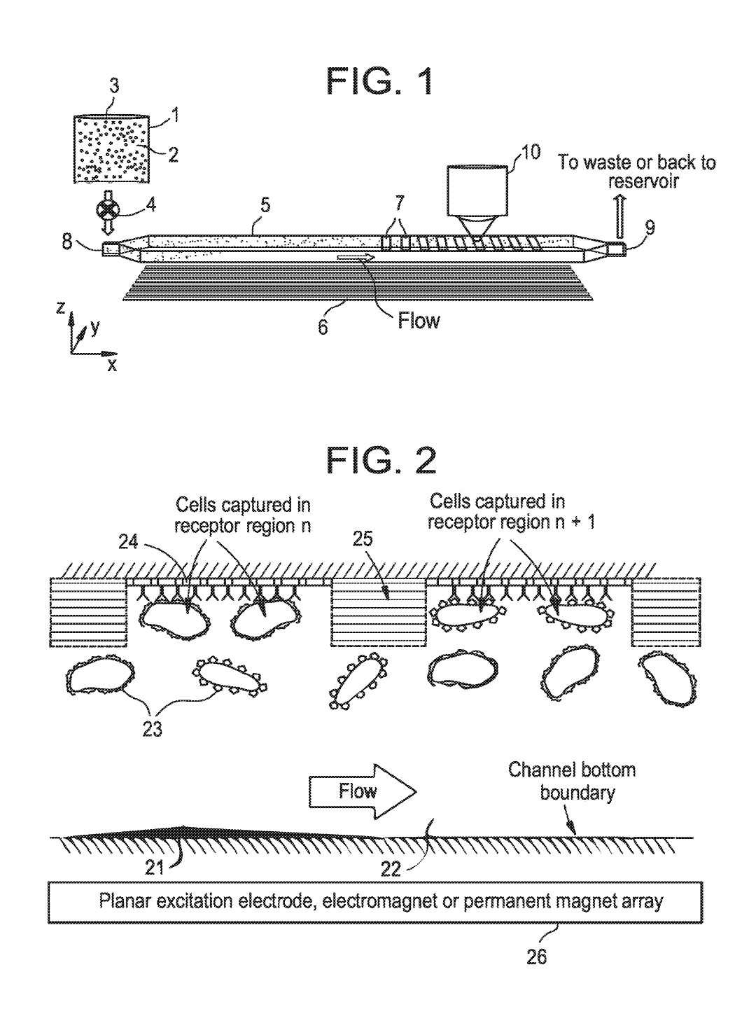

FIG. 1 illustrates a drug susceptibility assay system, utilizing a ferrofluid, according to some embodiments. As shown, an initial sample containing a mixture of cells 2 is mixed with a ferrofluid 3 (e.g., a biocompatible ferrofluid) in a reservoir 1. An external force, such as a pressure source, e.g., a pump 4, introduces the overall mixture into a channel inlet 8 that is connected to fluidic channel 5 that sits atop a magnetic field source 6. The magnetic field source 6, which is configured to apply a force, either directly or indirectly to particles/cells 2 of the mix (e.g., on non-magnetic particles/cells), such that the cells 2 are forced upward and focused. The magnetic source 6 may comprise at least one of a planar electrode(s), an electromagnet(s) and a permanent magnet(s), each of which may be arranged in an array. In some embodiments, the cells move along the channel ceiling (e.g., roll) and interact serially with receptor regions 7 on that surface. Specific interactions between the cells 2 and the receptor regions 7 result in the temporary, and in some embodiments permanent, capture of cells. In some embodiments, the captured cells are allowed to seed and grow over a predetermined period of time, for establishing their viability.

Thereafter, according to some embodiments, a predetermined dose of a drug is introduced into the flow, and the growth rate of the cells (or their morbidity) in response to the drug exposure is determined by a detecting means. In some embodiments, detecting means, such as an optical scanner 10, may be provided and configured to detect the target particles captured and/or moving along the receptor regions 7, such detecting means may also comprise, in addition or in place of, one or more of, for example: U.S. Pat. No. 4,448,534, WO2013/155525, WO2008/042003, U.S. Pat. No. 8,364,409, WO1991/001381, WO2013/054311 (as well as other detecting means familiar to those of skill in the art. The optical scanner may be, in some embodiments, impedance sensors, quartz crystal microbalance (QCM) sensors or surface plasmon resonance (SPR) sensors. The mixture flows through to the channel outlet 9, in some embodiments, to waste or back to the reservoir 1.

FIG. 2 illustrates a drug susceptibility assay utilizing a ferrofluid according to some embodiments. An initial sample containing a mixture of cells 23 mixed with ferrofluid 22 is configured to flow through a fluidic channel 21 that sits atop a magnetic field source 26 (for example). The magnetic field source 26, which may be configured to apply a force, either directly or indirectly to particles/cells 23 of the mix (e.g., on non-magnetic particles/cells), such that the cells 23 are forced upward and focused. The magnetic source 26 may comprise at least one of a planar electrode(s), an electromagnet(s) and a permanent magnet(s), each of which may be arranged in an array. In some embodiments, the cells move along the channel ceiling (e.g., roll) and interact serially with receptor regions 24 on that surface. In some embodiments, hydrodynamic barrier 25 is placed between receptor regions. Specific interactions between the cells 23 and the receptor regions 24 result in the temporary, and in some embodiments permanent, capture of cells.

This invention discloses a system and a method that combines the sensitivity of an impedance sensor or QCM with the sample manipulation, isolation and capture capability of a ferrofluidic device. A complex sample (such as whole blood) is mixed with a biocompatible ferrofluid and is introduced into a disposable cartridge that is placed on top of a magnetic field source (integrated current-carrying electrodes on a printed circuit board or its combination with other magnetic sources). As the ferrofluid-sample mixture is circulated within the fluidic network of the disposable cartridge, rare target cells or pathogens are separated, sorted, extracted, focused and directed towards the sensor surface that is functionalized with antibodies or other receptors corresponding to the specific cell or pathogen of interest. The target moieties are strongly pushed towards the sensor surface and are rapidly captured. The sensor channel is then flushed continuously with culture media and kept at an optimal culture temperature via thermal management hardware surrounding the cartridge.

Initially, the culture is allowed to grow (for up to 1 hour) to ensure that there are viable organisms captured over the sensor surface. Afterwards, an appropriate dosage of drug is introduced into the media. If the cells change the rate of their growth or stop dividing altogether, the susceptibility to the introduced drug may be quantified from the changes in the growth curve of those cells.

The signal from the functionalized sensor is taken differentially with respect to a non-functionalized sensor of matching geometry within the same channel. As the cells grow and divide, their total volume increases, leading to an increased differential signal on the impedance sensor. Similarly, if a QCM is used, changes in the total mass of all cells bound to the functionalized surface result in the signal.

Impedance and QCM based sensor geometries have been used to characterize cell cultures and/or determine drug susceptibility in the past. The advantage of the present invention is that it can extract and capture cells directly from a complex and large-volume sample without any additional sample preparation or pre-culture steps. Hence, within 2 hours of sample collection, drug susceptibility testing may be completed within this platform.

Another major advantage of this invention is its multiplexing capability. Bioferrofluidic sample extraction and purification is quite rapid, and the purified cells may be directed towards a very small sensor surface with great accuracy within the same cartridge. Hence, dozens of sensor surfaces may be used within a single cartridge to run simultaneous drug susceptibility tests of many different cell species.

Any and all references to publications or other documents, including but not limited to, patents, patent applications, articles, webpages, books, etc., presented in the present application, are herein incorporated by reference in their entirety.

Example embodiments of the devices, systems and methods have been described herein. As noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and devices which may further include any and all elements from any other disclosed methods, systems, and devices, including any and all elements corresponding to drug discovery and susceptibility. In other words, elements from one or another disclosed embodiments may be interchangeable with elements from other disclosed embodiments. In addition, one or more features/elements of disclosed embodiments may be removed and still result in patentable subject matter (and thus, resulting in yet more embodiments of the subject disclosure). Correspondingly, some embodiments of the present disclosure may be patentably distinct from one and/or another reference by specifically lacking one or more elements/features. In other words, claims to certain embodiments may contain negative limitation to specifically exclude one or more elements/features resulting in embodiments which are patentably distinct from the prior art which include such features/elements.

1-22. (canceled)

23. A system for determining drug effectiveness on a plurality of cells comprising:
a cartridge comprising a plurality of micro-channels;
an inlet receiving a ferrofluid and biological cell mixture, the inlet in communication with at least one of the plurality of micro-channels;
a magnetic field source arranged proximate to at least one of the inlet and the plurality of micro-channels;
at least one receptor region having receptors for binding with at least a first type of biological cell, the at least one receptor region arranged proximate to a first portion of at least one of the micro-channels, wherein the first portion is downstream from the inlet;
at least one quartz-crystal-microbalance (QCM) sensor arranged proximate to the first portion of at least one of the micro-channels; and
a controller;
wherein:
the magnetic field source generates a magnetic field that applies a force on the mixture so as to separate at least biological cells of the first type from the mixture,
at least a first micro-channel of the plurality of micro-channels receives biological cells of the first type and directs the first type of cells to the at least one receptor region,
each of the plurality of micro-channels receives a dosage of a drug to interact with the first type of biological cell captured by the receptors, and
the controller has computer instructions operating thereon to cause the controller to determine, using the at least one QCM sensor:
a first mass corresponding to the first type of cells captured by the receptors before the drug is received, and
a second mass corresponding to the first type of cells after the drug interacts with the first type of cells captured by the receptors.

24. The system of claim 23, wherein separating at least the biological cells of the first type from the mix comprises at least one of separating, focusing and concentrating.

25. The system of claim 23, wherein
the at least one receptor region comprises a plurality of receptor regions, each receptor region being functionalized with a specific receptor for at least one particular type of biological cell and each receptor corresponding to a specific micro-channel of the plurality of micro-channels; and
the magnetic field applies a force on the biological cells in the mix to separate a plurality of types of biological cells from the mix and direct the types of cells into one and/or another micro-channel.

26. The system of claim 23, wherein the first type of biological cell comprises a biological cell of a predetermined size, shape, weight, charge and/or configuration.

27. The system of claim 23, further comprising thermal managing means surrounding at least one of the cartridge, the first micro-channel, and the remainder of the micro-channels to substantially maintain the micro-channels at a first temperature.

28. The system of claim 23, wherein the drug is received by at least one of: the cartridge, the inlet, and a second inlet into one or more of the plurality of micro-channels.

29. The system of claim 23, wherein the controller has computer instructions operating thereon to cause the controller to determine, based on the first mass and the second mass, a susceptibility of the first type of cells to the first drug.

30. The system of claim 29, wherein the controller has computer instructions operating thereon to determine, based on the first mass and the second mass, the cell growth rate of the first type of cells.

31. The system of claim 30, wherein the controller has operating thereon computer instructions to cause the controller to track a signal of the at least one QCM sensor, such that an increase in mass corresponds to an increase in the total cell volume of the first type of cells and tracks changes in the total mass of the cells bound to the surface.

32. The system of claim 23, wherein the controller has computer instructions operating thereon to cause the controller to determine:
a first cell growth rate of the captured first type of cells before the drug is received, and
a second cell growth rate of the captured first type of cells after the drug interacts with the captured first type of cells.

33. The system of claim 32, wherein the controller has computer instructions operating thereon to cause the controller to determine susceptibility of the first type of cells to the drug by comparing the first cell growth rate and the second cell growth rate.
